(19) United States
(12) Patent Application Publication
(10) Pub. No.: US 2010/0253207 A1
(43) Pub. Date: Oct. 7, 2010

(54) FLAT UV DISCHARGE LAMP, USES AND MANUFACTURE

(75) Inventors: Laurent Joulaud, Soisy Sous Montmorency (FR); Guillaume Auday, Bussiere Saint Georges (FR); Didier Duron, Boulogne Billancourt (FR); Jingwei Zhang, Massy (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: SAINT-GOBAIN GLASS FRANCE, Courbevoie (FR)

(21) Appl. No.: 12/596,305

(22) PCT Filed: Apr. 17, 2008
(86) PCT No.: PCT/FR08/50694

§ 371 Date: Mar. 10, 2010

(30) Foreign Application Priority Data
Apr. 17, 2007 (FR) 0754533

(51) Int. Cl. H01J 61/38; H01J 9/02

(52) U.S. Cl. 313/484; 445/52

(57) ABSTRACT

The invention relates to a flat lamp (1) transmitting radiation in the ultraviolet, known as a UV lamp, comprising:
first and second flat dielectric walls (2, 3) that are facing each other, kept substantially parallel, and sealed, to one another, thus defining an internal space (10) filled with gas (7), the first dielectric wall at least being made of a material that transmits said UV radiation;
electrodes composed of first and second electrodes (4, 5), having different given potentials, for a perpendicular discharge between the walls, the first electrode at least being based on a layer arranged in order to allow overall UV transmission; and
an emitting gas or a phosphor coating (6) on one main inner face (22, 32) of the first and/or the second dielectric wall (2, 3), the phosphor emitting said UV radiation by being excited by the gas.
The invention also relates to the uses thereof and to the manufacture thereof.

The present invention relates to the field of flat UV (ultraviolet) lamps and in particular it relates to flat UV discharge lamps and to the uses of such UV lamps and to the manufacture thereof.

Conventional UV lamps are formed by UV fluorescent tubes filled with mercury and placed side by side in order to form an emitting surface. These tubes have a limited lifetime. Furthermore, the uniformity of the UV radiation emitted is difficult to obtain for large areas. Finally, such lamps are heavy and bulky.

Document U.S. Pat. No. 4,945,290 describes a flat UV discharge lamp that transmits two-directional UV radiation, comprising:
first and second flat walls, made of sapphire or quartz, kept substantially parallel, and sealed, to one another, thus defining an internal space filled with a gas that is a source of the UV radiation; and
two electrodes in the form of metal grids integrated into the quartz or on the main outer faces of the first and second flat walls and at different given potentials for a perpendicular discharge between the walls.

Document U.S. Pat. No. 4,983,881 describes a similar flat UV lamp with phosphor coatings on the main inner faces of the first and second dielectric walls, the phosphor emitting said UV radiation by being excited by the plasma gas.

One subject of the invention is to provide a flat UV discharge lamp that is of reliable performance, of simpler design and/or alternating operation preferably, and that is easy to produce, for a wide range of applications.

For this purpose, the invention provides a flat discharge lamp transmitting radiation in the ultraviolet (UV), comprising:
first and second flat dielectric walls that are facing each other, kept substantially parallel, and sealed, to one another, thus defining an internal gas-filled space, the first wall at least being made of a material that transmits said UV radiation;
first and second electrodes, at different given potentials, for a perpendicular discharge between the walls (“non-coplanar configuration”);
a first electrode on the outer main face of the first dielectric wall, the first electrode at least being a discontinuous layer thus arranged to allow an (optimal) overall UV transmission;
a second electrode integrated into the second dielectric wall or on the main outer face of the second dielectric wall; and
a source of the UV radiation comprising the gas and/or a phosphor coating on an inner main face of the first and/or of the second dielectric wall, the phosphor emitting said UV radiation by being excited by the gas.

The flat discharge lamp according to the invention is simpler to manufacture and gives access, in particular, to opaque materials in order to make the first electrode and preferably the second electrode.

The use of a discontinuous layer (single layer or multilayer) makes it possible to adjust or even improve the transmission threshold so as, in particular, to increase the uniformity.

The first electrode (and preferably the second electrode) may be discontinuous, by forming discontinuous (spaced apart from one another) electrode zones and/or by being an electroconducting layer with zones without the layer (insulating zones). It is possible to form a one-dimensional or two-dimensional array of zones of electrodes (arranged in lines, strips, a grid, etc.).

The UV lamp according to the invention may have dimensions of the order of those currently achieved with fluorescent tubes, or even greater, for example with an area of at least 1 m².

Preferably, the transmission factor of the lamp according to the invention about the peak of said UV radiation may be greater than or equal to 50%, more preferably still greater than or equal to 70%, and even greater than or equal to 80%.

The lamp must be hermetically sealed, the peripheral sealing may be achieved in various ways:
by a seal (polymeric seal of silicone type or else mineral seal of glass frit type); and
by a peripheral frame linked to the walls (by bonding or by any other means, for example a film based on a glass frit), for example made of glass.

The frame may optionally act as a spacer, replacing one or more of the individual spacers.

The dielectric walls act as a capactive protection for the electrodes against ion bombardment.

Each electrode may be associated with the outer face of the dielectric wall in question in various ways: it may be directly deposited on the outer face (preferred solution for the first electrode) or be on a dielectric bearing element, which is joined to the wall so that the electrode is pressed against its outer face.

This dielectric bearing element, which is preferably thin, may be a plastic film, in particular a lamination interlayer with a glass backing for mechanical protection, or a dielectric sheet for example bonded by a resin or a mineral seal preferably at the periphery in order to allow UV to pass through where appropriate.

Suitable plastics are, for example:
polyurethane (PU) used soft, ethylene/vinyl acetate copolymer (EVA) or polyvinyl butyral (PVB), these plastics serving as lamination interlayer, for example with a thickness between 0.2 mm and 1.1 mm, especially between 0.3 and 0.7 mm, optionally bearing an electrode (preferably the second electrode);
rigid polyurethane, polycarbonates, acrylates such as polymethyl methacrylate (PMMA), used especially as rigid plastic, optionally bearing an electrode (preferably the second electrode).

It is also possible to use PE, PEN or PVC or else polyethylene terephthalate (PET), the latter possibly being thin, especially between 10 and 100 μm, and possibly bearing the second electrode.

Where appropriate, it is necessary to ensure, of course, compatibility between various plastics used, especially as regards their good adhesion.

Of course, any dielectric element added is chosen to transmit said UV radiation if it is placed on an emission side of the UV lamp.

The UV radiation may be transmitted via a single side: the first wall. In this case, it is possible to choose a second electrode that forms a fully reflective UV layer and/or a second dielectric wall that absorbs the UV radiation and preferably has an expansion coefficient similar to the first wall. It is also possible to choose any type of electrode material (opaque or not) for example a wire electrode or an electrode having a layer inserted in a lamination of the second wall with a glass backing or a rigid plastic.

Preferably, the UV radiation may be two-directional, of the same intensity or of different intensity from the two sides of the lamp.

In order to make savings in the compactness, in the manufacturing time and/or in the UV transmission, the first (and preferably the second electrode chosen in the form of a layer) may be preferably deposited (directly) on the outer face and not be covered by a dielectric (especially by a dielectric (film, etc.) that covers the surface).

It is optionally possible to provide a discontinuous protective overlayer (for example a dielectric protective overlayer), superposed on the layer.

It is optionally possible to provide a functional underlayer (for example a dielectric, barrier, tie, etc. functional underlayer) underneath the electrode layer, that is preferably discontinuous and that is provided in a manner similar to the electrode layer.

With an electrode material that transmits said UV radiation, it is of course possible to increase the transmission via the discontinuities of the layer. It may especially be a very thin layer of gold, for example of the order of 10 nm, or of alkali metals such as potassium, rubidium, cesium, lithium or potassium, for example of 0.1 to 1 μm, or else be made of an alloy, for example with 25% sodium and 75% potassium.

The electrode material is not necessarily sufficiently transparent to UV radiation. One electrode (first and preferably second electrode) material that is relatively opaque to said UV radiation is, for example:
fluorine-doped tin oxide (SnO₂:F), or antimony-doped tin oxide, zinc oxide doped or alloyed with at least one of the following elements: aluminum, gallium, indium, boron, tin (for example ZnO:Al, ZnO:Ga, ZnO:In, ZnO:B, ZnSnO); and
indium oxide doped or alloyed in particular with zinc (IZO), gallium and zinc (IGZO) or tin (ITO),
the conductive oxides are, for example, deposited under vacuum,
a metal: silver, copper or aluminum, gold, molybdenum, tungsten, titanium, nickel, chromium or platinum.

The layer forming the first and preferably second electrode may be deposited by any known deposition means, such as liquid depositions, vacuum depositions (sputtering, especially magnetron sputtering, evaporation), by pyrolysis (powder or gas route) or by screen printing, by an inkjet, by applying with a doctor blade or more generally by printing.

One electrode (first electrode and preferably second electrode) material that is relatively opaque to said UV radiation is, for example, based on metallic particles or conductive oxides, for example those already cited.

It is possible to choose nanoparticles that are therefore of nanoscale size (for example with a maximum nanoscale dimension and/or a nanoscale D50), especially having a size between 10 and 500 nm, or even less than 100 nm to facilitate the deposition/formation of thin features (for a sufficient overall transmission for example), especially by screen printing.

As metallic (nano)particles (sphere, flake, etc.) it is possible to choose, in particular, (nano)particles based on Ag, Au, Al, Pd, Pt, Cr, Cu, Ni.

The (nano)particles are preferably in a binder. The resistivity is adjusted for the concentration of (nano)particles in a binder.

The binder may optionally be organic, for example polyurethane, epoxy or acrylic resins, or be produced by the sol-gel process (mineral, or hybrid organic-inorganic, etc.).

The (nano)particles may be deposited from a dispersion in a solvent (alcohol, ketone, water, glycol, etc.).

Commercial products based on particles that may be used to form the first and/or the second electrode are the products sold by Sumitomo Metal Mining Co. Ltd. below:
X100®, X100®D particles of ITO dispersed in a resin binder (optional) and with a ketone solvent;
X500® particles of ITO dispersed in an alcohol solvent;
CKR® particles of gold-coated silver in an alcohol solvent;
CKRF® agglomerated particles of gold and of silver.

The desired resistivity is adjusted as a function of the formulation.

Particles are also available from Cabot Corporation USA (e.g. Product No. AG-IJ-G-100-S1) or from Harima Chemicals, Inc. in Japan (NP series).

Preferably, the particles and/or the binder are essentially inorganic.

For the first electrode and preferably for the second electrode (especially if two-directional radiation is desired) it is possible to choose:
a screen-printing paste, especially:
a paste filled with (nano)particles (such as already cited, preferably silver and/or gold): a conductive enamel (a silver fused glass frit), an ink, a conductive organic paste (having a polymer matrix), a PSS/PEDOT (from Bayer, Agfa) and a polyaniline,
a sol-gel layer with (metallic) conductive (nano) particles that precipitate after printing; and
a conductive ink filled with (nano)particles (such as already cited, preferably silver and/or gold) deposited by inkjet, for example the ink described in document US 2007/0283848.

Preferably, the first electrode (and the second electrode) is essentially inorganic.

The arrangement of the first electrode (and, preferably of the second electrode where appropriate) may be obtained directly by deposit(s) of electrically conductive material(s) in order to reduce the manufacturing costs. Thus, post-structuring operations are avoided, for example dry and/or wet etching operations, that often require lithographic processes (exposure of a resist to a radiation and development).

This direct arrangement as an array may be obtained directly by one or more suitable deposition methods, preferably a deposition via a liquid route, via printing, especially flat or rotary printing, for example using an ink pad, or else via an inkjet (with a suitable nozzle), via screen or silk printing or by simple application with a doctor blade.

Via screen or silk printing, a synthetic, silk, polyester or metallic cloth with a suitable mesh width and a suitable mesh fineness is chosen.

The first and/or the second electrode may be thus principally in the form of a series of equidistant strips, which may be connected by an especially peripheral strip for a common electrical power supply. The strips may be linear, or be of more complex, nonlinear, shapes, for example angled, V-shaped, corrugated or zigzagged.

The strips may be linear and substantially parallel, having a width l1 and being spaced a distance d1 apart, the ratio l1 to d1 possibly being between 10% and 50%, in order to allow an overall UV transmission of at least 50%, the l1/d1 ratio possibly also being adjusted as a function of the transmission of the associated wall.

More broadly, the first and/or the second electrode may be at least two series of strips (or lines) which are overlapped, for example organized as a woven fabric, cloth or grid.

For example, for all the series of strips, the same strip size and spacing between adjacent strips is chosen.

Furthermore, each strip may be solid or of open structure.

For the second electrode, the solid strips may especially be formed from contiguous conducting wires (parallel wires, braided wires, etc.) or from a ribbon (made of copper, to be bonded, etc.).

The solid strips may be from a coating deposited by any means known to a person skilled in the art such as liquid depositions, vacuum depositions (magnetron sputtering, evaporation), by pyrolysis (powder or gas route) or by screen printing.

To form strips in particular, it is possible to employ masking systems in order to attain the desired distribution directly, or else to etch a uniform coating by laser ablation or by chemical or mechanical etching.

Each strip having an open structure may also be formed from one or more series of conductive features, forming an array. The feature is especially geometrical and elongate or not (square, round, etc.).

Each series of features may be defined by equidistant features, with a given pitch known as p1 between adjacent features and a width known as l2 of the features. Two series of features may be overlapped. This array may especially be organized as a grid, such as a woven fabric, a cloth. These features are, for example, made of metal such as tungsten, copper or nickel.

Each strip having an open structure may be based on conductive wires (for the second electrode) and/or conductive tracks.

Thus, it is possible to obtain an overall UV transmission by adapting the l1 to d1 ratio of the one or more series of strips as a function of the desired transmission and/or by adapting, as a function of the desired transmission, the width l2 and/or the pitch p1 of strips having an open structure.

Thus, the ratio of the width l2 to the pitch p1 may preferably be less than or equal to 50%, preferably less than or equal to 10%, more preferably still less than or equal to 1%.

For example, the pitch p1 may be between 5 μm and 2 cm, preferably between 50 μm and 1.5 cm, more preferably still 100 μm and 1 cm, and the width l2 may be between 1 μ and 1 mm, preferably between 10 and 50 μm.

By way of example, it is possible to use an array of conductive tracks (as a grid, etc.) with a pitch p1 between 100 μm and 1 mm, or even 300 μm, and a width l2 of 5 μm to 200 μm, less than or equal to 50 μm, or even between 10 and 20 μm.

An array of conductive wires for the second electrode may have a pitch p1 between 1 and 10 mm, in particular 3 mm, and a width l2 between 10 and 50 μm, in particular between 20 and 30 μm.

For the second electrode, the wires may be at least partly integrated into the second associated dielectric wall, or alternatively at least partly integrated into a lamination interlayer, especially made of PVB or PU.

When the gas is a UV source, then in order to change the UV radiation, the gas must be replaced and it is then necessary to adapt the UV emission and discharge conditions (pressure, supply voltage, gas height, etc.) as a consequence.

If the phosphor coating(s) is (are) chosen as a function of the UV radiation(s) that it is desired to produce, independently of the discharge conditions, it is therefore not necessary to change the excitation gas.

In particular, phosphors exist that emit in the UVC when exposed to VUV radiation, for example produced by one or more noble gases (Xe, Ar, Kr, etc.). For example, UV radiation at 250 nm is emitted by phosphors after being excited by VUV radiation shorter than 200 nm. Mention may be made of materials doped with Pr or Pb such as: LaPO₄:Pr, CaSO₄:Pb, etc.

Phosphors also exist that emit in the OVA or near UVB also when exposed to VUV radiation. Mention may be made of gadolinium-doped materials such as YBO₃:Gd; YB₂O₅:Gd; LaP₃O₉:Gd; NaGdSiO₄; YAl₃(BO₃)₄:Gd; YPO₄Gd; YAlO₃:Gd; SrB₄O₇:Gd; LaPO₄:Gd; LaMgB₅O₁₀:Gd,Pr; LaB₃O₈:Gd,Pr; (CaZn)₃(PO₄)₂:Tl.

In addition, phosphors exist that emit in the UVA when exposed to UVB or UVC radiation, for example produced by mercury or preferably one (some) gas(es) such as noble and/or halogen gases (Hg, Xe/Br, Xe/I, Xe/F, Cl₂, etc.). Mention may be made, for example, of LaPO₄:Ce; (Mg,Ba) Al₁₁O₁₉:Ce; BaSi₂O₅:Pb; YPO₄:Ce; (Ba,Sr,Mg)₃Si₂O₇:Pb; SrB₄O₇:Eu. For example, UV radiation above 300 nm, especially between 318 nm and 380 nm, is emitted by phosphors after being excited by UVC radiation of around 250 nm.

Thus, the gas may consist of a gas or a mixture of gases chosen from noble gases and/or halogens. The amount of halogen (as a mixture with one or more noble gases) may be chosen to be less than 10%, for example 4%. It is also possible to use halogenated compounds. The noble gases and the halogens have the advantage of being unaffected by climatic conditions.

Table 1 below indicates the radiation peaks of the UV-emitting and/or excitation gases of the phosphors.

TABLE 1

| Phosphor UV-emitting and/or | |
|---|---|
| excitation gases | Peak(s) (nm) |
| Xe | 172 |
| F₂ | 158 |
| Br₂ | 269 |
| C | 259 |
| I₂ | 342 |
| XeI/KrI | 253 |
| ArBr/KrBr/XeBr | 308/207/283 |
| ArF/KrF/XeF | 351/249/351 |
| ArCl/KrCl/XeCl | 351/222/308 |
| Hg | 185, 254, 310, 366 |

More preferably still, one or more noble gases, especially xenon, will be chosen as the excitation gas.

Naturally, in order to maximize the discharge zone and for a uniform discharge, the first and second electrodes, continuously or in pieces, may extend over areas having dimensions at least substantially equal to the area of the walls inscribed in the internal space.

For greater simplicity and to facilitate the sealing, the first and second dielectric walls may be made of identical materials or materials at least having a similar expansion coefficient.

The material that transmits said UV radiation from the first or even from the second dielectric wall may preferably be chosen from quartz, silica, magnesium fluoride (MgF₂) or calcium fluoride (CaF₂), a borosilicate glass, or a soda-lime-silica glass, especially with less than 0.05% of Fe₂O₃.

As examples for thicknesses of 3 mm:
the magnesium or calcium fluorides transmit more than 80%, or even 90%, over the entire range of UV bands, that is to say UVA (between 315 and 380 nm), UVB (between 280 and 315 nm), UVC (between 200 and 280 nm) or VUV (between around 10 and 200 nm);
quartz and certain high-purity silicas transmit more than 80%, or even 90%, over the entire range of UVA, UVB and UVC bands;
borosilicate glass, such as Borofloat from Schott, transmits more than 70% over the entire UVA band; and
soda-lime-silica glasses with less than 0.05% of Fe(III) or of Fe₂O₃, especially the Diamant glass from Saint-Gobain, the Optiwhite glass from Pilkington, the B270 glass from Schott, transmit more than 70%, or even 80%, over the entire UVA band.

A soda-lime-silica glass, such as the Planilux glass sold by Saint-Gobain, has a transmission greater than 80% above 360 nm which may be sufficient for certain constructions and certain applications.

In the structure of the flat UV lamp according to the invention, the gas pressure in the internal space may be around 0.05 to 1 bar.

The dielectric walls may be of any shape: the contour of the walls may be polygonal, concave or convex, especially square or rectangular, or curved, especially round or oval.

The dielectric walls may be slightly curved, with the same radius of curvature, and are preferably kept a constant distance apart, for example by a spacer (for example a peripheral frame) or spacers (point spacers, etc.) at the periphery or preferably distributed (regularly, uniformly) in the internal space. For example, they may be glass beads. These spacers, which may be termed discrete spacers when their dimensions are considerably smaller than the dimensions of the glass walls, may take various forms, especially in the form of spheres, parallel-faced bitruncated spheres, cylinders, but also parallelepipeds of polygonal cross section, especially cruciform cross section, as described in document WO 99/56302.

The gap between the two dielectric walls may be fixed by the spacers at a value of around 0.3 to 5 mm. A technique for depositing the spacers in vacuum insulating glazing units is known from FR-A-2 787 133. According to this process, spots of adhesive are deposited on a glass plate, especially spots of enamel deposited by screen printing, with a diameter equal to or less than the diameter of the spacers, and then the spacers are rolled over the glass plate, which is preferably inclined, so that a single spacer adheres to each spot of adhesive. The second glass plate is then placed on the spacers and the peripheral sealing joint is deposited.

The spacers are made of a nonconducting material in order not to participate in the discharges or to cause a short circuit. Preferably, they are made of glass, especially of the soda-lime type. To prevent light loss by absorption in the material of the spacers, it is possible to coat the surface of the spacers with a material that is transparent or reflective in the UV, or with a phosphor material identical to or different from that used for the wall(s).

According to one embodiment, the UV lamp may be produced by manufacturing firstly a sealed enclosure in which the intermediate air cavity is at atmospheric pressure, then by creating a vacuum and by introducing the plasma gas at the desired pressure. According to this embodiment, one of the walls includes at least one hole drilled through its thickness and obstructed by a sealing means.

The UV lamp may have a total thickness of less than or equal to 30 mm, preferably less than or equal to 20 mm.

Preferably, the walls are sealed by a peripheral sealing joint which is inorganic, for example based on a glass frit.

The first electrode may be at a potential lower than the second electrode, especially in a configuration with one emitting side, the second electrode possibly then being protected by dielectric.

The first electrode may be at a potential less than or equal to 400 V (typically peak voltage), preferably less than or equal to 220 V, more preferably still less than or equal to 110 V and/or at a frequency f which is less than or equal to 100 Hz, preferably less than or equal to 60 Hz and more preferably still less than or equal to 50 Hz.

V1 is preferably less than or equal to 220 V and the frequency f is preferably less than or equal to 50 Hz.

The first electrode may preferably be grounded.

The power supply of the UV lamp may be alternating, periodic, especially sinusoidal, pulsed, or a crenellated (square-wave, etc.) signal.

The UV lamp as described above may be used both in the industrial sector, for example in the beauty, electronics or food fields, and in the domestic sector, for example for decontaminating tap water, drinking water or swimming pool water, air, for UV drying and for polymerization.

By choosing radiation in the UVA or even in the UVB, the UV lamp as described above may be used:
as a tanning lamp (especially 99.3% in the UVA and 0.7% in the UVB according to the standards in force) especially built into a tanning booth;
for photochemical activation processes, for example for polymerization, especially of adhesives, or crosslinking or for drying paper;
for the activation of fluorescent material, such as ethidium bromide used in gel form, for analyzing nucleic acids or proteins; and
for activating a photocatalytic material, for example for reducing odors in a refrigerator or dirt.

By choosing radiation in the UVB, the lamp promotes the formation of vitamin D in the skin.

By choosing radiation in the UVC, the UV lamp as described above may be used for disinfecting/sterilizing air, water or surfaces, by a germicide effect, especially between 250 nm and 260 nm.

By choosing radiation in the far UVC or preferably in the VUV for ozone production, the UV lamp as described above is used especially for the treatment of surfaces, in particular before the deposition of active films for electronics, computing, optics, semiconductors, etc.

The lamp may for example be integrated into household electrical equipment, such as a refrigerator or kitchen shelf.

Another subject of the invention is the process for manufacturing a UV lamp, especially of the type of that described previously, in which a discontinuous electrode (first electrode and/or second electrode) is formed for an overall UV transmission directly by liquid deposition on the main face of a dielectric wall and the arrangement of the is formed directly by liquid deposition on the outer face (coated with an underlayer or not) of the first wall.

In particular, a printing technique is preferred (flexography, pad printing, roller printer, etc.) and especially screen printing and/or inkjet printing.

Furthermore, a peripheral electrical power supply zone of the electrodes is generally formed. This zone, for example that forms a strip, is known as a “busbar”, and is itself connected, for example by brazing or welding, to a power supply means (via a foil, a wire, a cable, etc.). This zone may extend along one or more sides.

This electric power supply zone may be screen printed, especially made of silver enamel.

Thus, it may be preferred to form at least one peripheral electrical power supply zone of the discontinuous electrode during the step of depositing said electrode by screen printing (preferably from a conductive enamel) or even by inkjet printing. This process for manufacturing the UV electrode is suitable for the UV lamp such as that described previously or for a UV lamp with electrodes on the inner faces, or else one on an inner face, the other on an outer face.

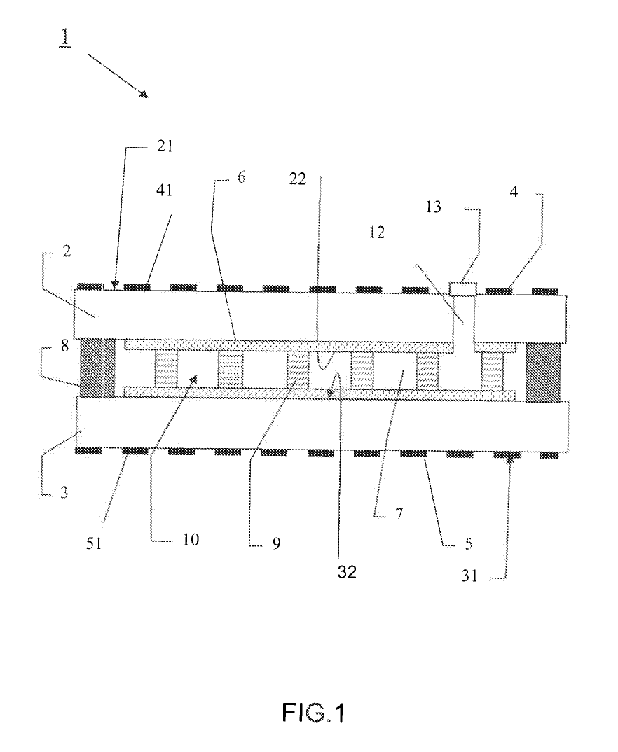

Other details and advantageous features of the invention will appear on reading the example of the flat UV lamp illustrated by FIG. 1 below which schematically represents a cross-sectional view of a flat UV discharge lamp in one embodiment of the invention.

It is stated that, for reasons of clarity, the various elements of the articles represented are not necessarily reproduced to scale.

FIG. 1 presents a flat UV discharge lamp 1 comprising first and second plates 2, 3, for example that are rectangular, each having an outer face 21, 31 and an inner face 22, 32. The lamp 1 emits two-directional UV radiation via its outer faces 21, 31.

The area of each plate 2, 3 is, for example, of the order of 1 m², or even greater, and their thickness is of the order of 3 mm.

The plates 2, 3 are joined together so that their inner faces 22, 32 face each other and are assembled by means of a peripheral seal that defines the internal space, here by a sealing frit 8, for example a glass frit having a thermal expansion coefficient close to that of the plates 2, 3.

As a variant, the plates are joined together by an adhesive, for example a silicone adhesive (that forms a seal) or else by a heat-sealed glass frame. These sealing modes are preferable if plates 2, 3 having excessively different expansion coefficients are chosen.

The gap between the plates is set (generally at a value of less than 5 mm) by glass spacers 9 placed between the plates. Here, the gap is for example between 1 and 2 mm.

The spacers 9 may have a spherical, cylindrical or cubic shape or another polygonal, for example cruciform, cross section. The spacers may be coated, at least on their lateral surface exposed to the plasma gas atmosphere, with a material that reflects the UV radiation.

The first plate 2 has, near the periphery, a hole 13 drilled through its thickness, with a diameter of a few millimeters, the external orifice of which is obstructed by a sealing pad 12, especially made of copper, welded to the outer face 21.

In the space 10 between the plates 2, 3 there is a reduced pressure of 200 mbar of xenon 7 in order to emit exciting radiation in the UVC.

The lamp 1 is used, for example, as a tanning lamp.

The inner faces 22, 32 bear a coating 6 of phosphor material which emits radiation in the UVA, preferably beyond 350 nm, such as YPO₄:Ce (peak at 357 nm) or (Ba,Sr,Mg)₃Si₂O₇:Pb (peak at 372 mm) or SrB₄O₇:Eu (peak at 386 nm).

A soda-lime-silica glass, such as Planilux sold by Saint-Gobain, is chosen, which gives a UVA transmission at around 350 nm of greater than 80% for low cost. Its expansion coefficient is around 90×10⁻⁸ K⁻¹.

In another variant, a gadolinium-based phosphor and a borosilicate glass (for example having an expansion coefficient of around 32×10⁻⁸ K⁻¹) or a soda-lime-silica glass with less than 0.05% of Fe₂O₃, and also a noble gas such as xenon, alone or as a mixture with argon and/or neon, are chosen.

Naturally, other phosphors and a borosilicate glass for transmitting UVA at around 300-330 nm may be chosen.

In another variant, the lamp 1 emits in the UVC, for a germicidal effect, then a phosphor such as LaPO₄:Pr or CaSO₄:Pb is chosen and for the walls silica or quartz are chosen and also a noble gas such as xenon, preferably alone or as a mixture with argon and/or neon is chosen.

The first electrode 4 is on the outer face 21 of the first wall 2 (always the emitting side). The second electrode 5 is on the outer face 31 of the second wall 3 (optionally emitting side).

Each electrode 4, 5 is in the form of a discontinuous layer at a unique potential. Each electrode 4, 5 is in the form of at least one series, or even two overlapped series, of strips 41, 51, for example solid strips.

Preferably, the strips 41, 51 have a width l1 and similar inter-strip spacings d1.

The material of the first electrode (at least) is relative opaque to UV, in which case the ratio of the width of the strips l1 to the width of the inter-strip space d1 is consequently adjusted in order to increase the overall UV transmission (for each series).

For example, a ratio of the width l1 to the width d1 of the inter-strip space is chosen of the order of 20% or less, for example the width l1 is equal to 4 mm and the width d1 of the inter-electrode space is equal to 2 cm.

The material of the electrode 4, 5 is for example silver preferably deposited by screen printing: for example a silver enamel or an ink with silver and/or gold nanoparticles.

The electrode material may alternatively be deposited as a thin film by sputtering and then be etched.

Thus, it is possible for example to choose the Planilux glass with a layer of copper, or silver or else fluorine-doped tin oxide which is etched in order to form the electrodes 4, 5 with a width equal to 1 mm and a space equal to 5 mm that makes it possible to obtain an overall transmission of 85% approximately starting from 360 nm, while retaining a very satisfactory uniformity.

It is also possible to choose, for the walls, Planilux glasses each with a layer of fluorine-doped tin oxide which is etched in order to form the electrodes 4, 5 with a width equal to 1 mm and a space equal to 5 mm that makes it possible to obtain an overall transmission of 85% approximately starting from 360 nm, while retaining a very satisfactory uniformity.

As a variant, each strip has an open structure (for example having a width of 15 to 50 μm and spaced 500 μm apart and produced by screen printing) and may, for example, be formed from an array of conductive features, for example geometrical features (square, round, etc. features, lines, grid), in order to further increase the overall UV transmission.

As a variant, the electrodes 4, 5 are discontinuous layers that extend over the faces and are arranged as a grid, for example having a width of the tracks between 15 and 50 μm and spaced 500 μm apart, produced by screen printing. For example, the TEC PA 030™ ink from InkTec Nano Silver Paste Inks is chosen or a silver-based glass frit is screen printed.

In another embodiment variant, the second electrode 5 is a solid layer of aluminum that forms a UV mirror.

In a last embodiment variant, the second electrode 5 is a grid integrated into the wall 3 or embedded into an EVA or PVB type lamination interlayer with a backing glass.

Each of the electrodes 4, 5 is powered by a flexible foil 11, 11′ or as a variant via a welded wire. The first electrode 4 is at a potential V0 of the order of 1100 V and has a frequency between 10 and 100 kHz, for example 40 kHz. The second electrode 5 is grounded.

Alternatively, the electrodes 4 and 5 are powered, for example, by signals that are in phase opposition, for example respectively at 550 V and −550 V.

The first electrode is preferably grounded and the second electrode powered by the high-frequency signal when a single side is an emitter. As a variant, the second electrode may then be protected.

The first electrode 4 may be electrically connected to a current supply strip (commonly known as a “busbar”) which covers the overlapped strips 51 (or the grid in the variant), at the periphery of at least one edge (for example a longitudinal edge) of the first wall 2 and onto which a wire or a foil is welded.

The second electrode 5 may be electrically connected to a current supply strip (commonly known as a “busbar”) which covers the overlapped strips (or the grid in the variant), at the periphery of at least one edge (for example a longitudinal edge) of the second wall and onto which a wire or a foil is welded.

These strips may be made of screen-printed silver enamel or be deposited by inkjet printing, especially at the same time as the electrodes (a solid peripheral and sufficiently large strip is thus provided).

1. A flat discharge lamp transmitting radiation in the ultraviolet (UV), comprising:
first and second flat dielectric walls that are facing each other, kept substantially parallel, and sealed, to one another, thus defining an internal space filled with gas, the first dielectric wall at least being made of a material that transmits said UV radiation;
first and second electrodes, at different given potentials, for a perpendicular discharge between the walls;
a first electrode on the outer main face of the first dielectric wall;
a second electrode integrated into the second dielectric wall or on the main outer face of the second dielectric wall; and
a source of the UV radiation comprising the gas and/or a phosphor coating on an inner main face of the first and/or of the second dielectric wall, the phosphor emitting said UV radiation by being excited by the gas,
wherein the first electrode is at least a discontinuous layer, arranged in order to allow overall UV transmission.

2. The UV lamp as claimed in claim 1, wherein the first electrode is deposited on the outer face and is not covered by a dielectric that covers the surface.

3. The UV lamp as claimed in claim 1, wherein the second electrode is a layer arranged in order to allow an overall UV transmission.

4. The UV lamp as claimed in claim 1, wherein the UV radiation is from two sides of the lamp.

5. The UV lamp as claimed in claim 1, wherein the first electrode is in the form of a series of equidistant strips or of at least two overlapped series of parallel strips, each strip having a width l1 and being spaced a distance d1 away from an adjacent strip, and in that the ratio l1 to d1 is between 10% and 50%.

6. The UV lamp as claimed in claim 1, wherein the second electrode is discontinuous, in the form of a series of equidistant strips, as a layer, or of at least two overlapped series of parallel strips, each strip having a width l1 and being spaced a distance d1 away from an adjacent strip, and in that the ratio l1 to d1 is between 10% and 50%.

7. The UV lamp as claimed in claim 1, wherein the first electrode and/or the second electrode is in the form of strips, each formed from one or more series of conductive features defined by a given pitch known as p1 between features and a width known as l2 of the features, the ratio of the width l2 to the pitch p1 being less than or equal to 50%.

8. The UV lamp as claimed in claim 1, wherein at least the first electrode is organized as a grid.

9. The UV lamp as claimed in claim 1, wherein at least the first electrode is based on conductive particles comprising silver and/or gold, optionally in a binder.

10. The UV lamp as claimed in claim 1, wherein at least the first electrode is a conductive enamel or a conductive ink containing silver and/or gold.

11. The UV lamp as claimed in claim 1, wherein the material transmitting said UV radiation is chosen from quartz, silica, magnesium or calcium fluoride, a borosilicate glass, a soda-lime-silica glass, comprising less than 0.05% of Fe₂O₃.

12. The UV lamp as claimed in claim 1, wherein the gas comprises a noble gas or a mixture of gases chosen from noble gases and halogen gases.

13. A UV lamp in the beauty, electronics or food fields comprising the UV lamp as claimed in claim 1.

14. A UV lamp as a tanning lamp, for dermatological treatment, for disinfecting or sterilizing surfaces, air, tap water, drinking water, or swimming pool water, for the treatment of surfaces before deposition of active layers, for activating a photochemical process of the polymerization or crosslinking type, for drying paper, for analyses starting from fluorescent materials, or for activation of a photocatalytic material comprising the UV lamp as claimed in claim 1.

15. A process for manufacturing a UV lamp, wherein a discontinuous electrode is formed for an overall UV transmission directly by liquid deposition on the main face of a dielectric wall.

16. The process for manufacturing the UV lamp as claimed in claim 15, wherein said electrode arrangement is formed by screen printing or by inkjet.

17. The process for manufacturing the UV lamp as claimed in claim 15, wherein at least one peripheral electrical power supply zone of the discontinuous electrode is formed during the step of deposition of the first electrode by screen printing or by inkjet.
